(19) United States
(12) Patent Application Publication
(10) Pub. No.: US 2023/0402161 A1
(43) Pub. Date: Dec. 14, 2023

(54) SMART LOCKER SYSTEM FOR ON-DEMAND MEDICAL SUPPLY

(71) Applicant: Medi-Sense Inc., Smyma, GA (US)

(72) Inventor: Eric L. Brown, Smyma, GA (US)

(21) Appl. No.: 18/209,736

(22) Filed: Jun. 14, 2023

Related U.S. Application Data
(60) Provisional application No. 63/352,121, filed on Jun. 14, 2022.

(51) Int. Cl. G16H 40/20; G07C 9/00; G07C 9/22; A61B 50/10

(52) U.S. Cl. CPC G16H 40/20; G07C 9/00309; G07C 9/22; A61B 50/10; G07C 2009/00388; A61B 2050/105

(57) ABSTRACT

A system for secure lockers for dispensing medical devices includes a plurality of lockers, each of the plurality of lockers having a respective door; an I/O device; and a controller in communication with each of the plurality of lockers and to the I/O device. The controller is configured to receive a code; process the code to retrieve a datapoint indicative of an order; command the I/O device to display a prompt for additional verifying information regarding the order; in response to receiving additional verifying information, determine a locker of the plurality of lockers associated with the order; and command the respective door of the determined locker to open.

### CROSS-REFERENCE TO RELATED APPLICATIONS

This application is a non-provisional conversion of U.S. Pat. App. No. 63/352,121 entitled “SMART LOCKER SYSTEM FOR ON-DEMAND MEDICAL SUPPLY,” filed Jun. 14, 2022, the contents of which are hereby incorporated in their entirety and for all purposes.

### TECHNICAL FIELD

This disclosure generally relates to a locker system for on-demand supply of medical devices.

### BACKGROUND

Based on cost and demand, some medical devices are stocked by providers for patients, while others are ordered on an as-needed basis.

### BRIEF DESCRIPTION OF THE DRAWINGS

FIG. 1 is an example embodiment of a secure locker system for medical devices.

FIG. 2 is a block diagram of a computing system that includes the secure locker system of FIG. 1.

FIG. 3 is a flow chart illustrating an example method of utilizing the secure locker system of FIG. 1.

FIG. 4 is a flow chart illustrating an example method of utilizing the secure locker system of FIG. 1.

### DETAILED DESCRIPTION

Known methods for distributing certain medical devices (e.g., long-acting reversible contraceptives, or LARC) introduce delays for patients to receive needed medical care. For example, in one known method, a medical provider may stock devices for distribution to the patient as needed. Under this method, referred to as “Buy & Bill,” a selection and stock of medical devices is generally limited.

Another method of providing such medical devices to patients relies on specialty pharmacies, which may offer a larger selection but typically delay the delivery of the medical devices to the patient.

As such, there is a need for a system that can securely store high-cost medical supplies in a way that is convenient for providers and patients to provide needed medical devices to patients in a timely manner.

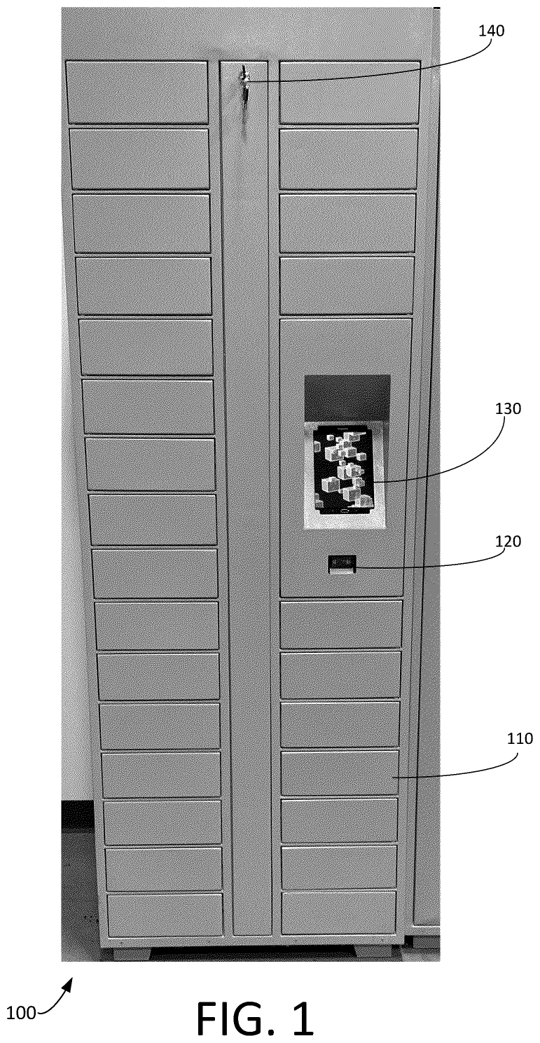

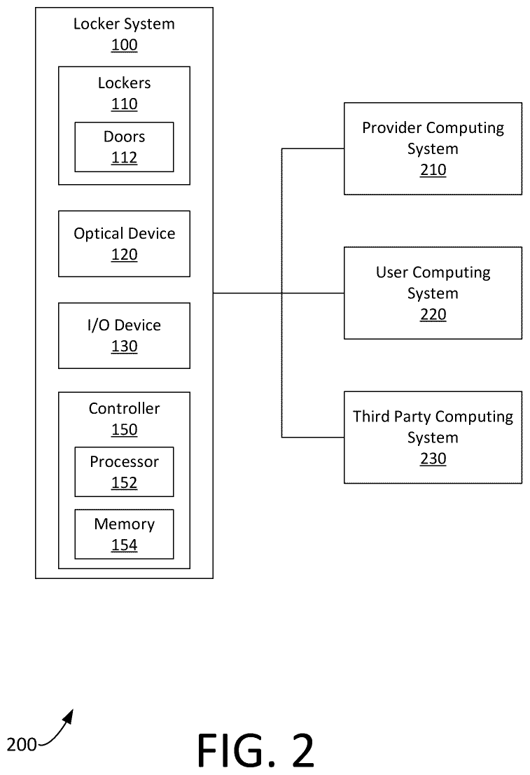

Referring now to the Figures, FIGS. 1 and 2 show a secure locker system 100, according to an example embodiment. As shown, the secure locker system 100 includes a plurality of lockers 110, an optical element 120, an I/O device 130, a master key 140, and a controller 150. The plurality of lockers 110 may be secured by selectively-locking doors 112, which may be controlled by the controller 150. The controller 150 may be any suitable backend controller having a memory 154 and a processor 152 configured to execute instructions stored in the memory 154, which may be any suitable computer-readable medium. The optical element 120 may be configured to scan for codes (e.g., QR codes, bar codes). Following a scan, the optical element 120 may communicate with the controller 150 to provide the controller 150 with the information from the scan, which the controller 150 may further process to derive actionable data from the scan. For example, the optical element 120 may scan a QR code that indicates a patient has been approved for a particular medical device. Upon scanning the QR code, the optical element 120 may transmit this approval to the controller 150, which then commands the correct locker 110 to open and provide access to the medical device. In a related example, the optical element 120 may scan the same QR code, but rather than processing the code as indicative of approval and then passing that approval to the controller 150, the optical element 120 may capture the QR code and send the substantially-raw captured data to the controller 150. In this example, then, the determination that the QR code is indicative of patient approval is made by the controller 150.

In yet another related example, the optical element 120 may scan this same QR code and may process the scan but only to determine relevance rather than to derive all encoded information (e.g., partially process). In this example, the optical element 120 may still send a substantially-raw datapoint to the controller 150, but will have first determined that the QR code is relevant to the machine. Alternatively, if the optical element 120 scans a different QR code that may have been incorrectly presented by a user (e.g., the user provides an outdated QR code or a QR code for a different machine), the optical element 120 may process the QR code scan enough to determine that the QR code is irrelevant, and may send an indication of the irrelevancy to the controller 150. The controller 150 may then, in this example, command the I/O device 130 to display a corresponding error message.

The optical element 120 may determine relevance by deriving a portion of the information that is associate with a domain, owner, originating IP address, responsible party, or other datapoint that may be relied upon by the optical element 120 to determine an origin of the QR code. To do so, the optical element 120 may (1) process the QR code to derive (e.g., extract) one or more labels (e.g., metadata) associated with each datapoint in the encoded information, (2) determine which of the datapoints relates to origin based on the derived labels, (3) further process that datapoint to derive the associated origin data, and (4) determine whether the QR code is relevant based on the origin data. In addition to origin data, the QR code may include information indicative of or related to a patient, an order, a medical device(s) included in the order, a provider that authorized the order, a cost of the order, and an insurance policy used to pay for the order. Each of these pieces of information may include a metadata label for facilitating extraction of specific datapoints.

In some embodiments, the I/O device 130 may directly receive a code (e.g., a numerical code) via a keypad on the I/O device 130, and may send this code to the controller 150 for further processing. The master key 140 (which may be a physical key or may be a particular code) may provide access to the entire plurality of lockers 110, and may be used by a stocker or supplier of the medical devices.

The controller 150 may take one or more actions in response to information received or derived from the code (e.g., QR code, entered code, master key 140, etc.). These actions may include presenting a message on the I/O device 130, prompting a user for information via the I/O device 130, and commanding a locker of the plurality of lockers to open. The presented message may be an error message if, for example, the scanned/entered code is invalid, inapplicable, or irrelevant, as described above. The presented message may be an indication of a locker 110 that the I/O device 130 may be concurrently commanding to open. The prompt may be a request for verifying information personal to the user, such as a birthday or Social Security Number, as an additional step for HIPAA compliance. The verifying information may be any piece of information that satisfies a “Something-You-Know” factor test, such that the verifying information—in combination with the “Something-You-Have” factor test satisfied by the code—may utilize two-factor authentication. The prompt may be a description of the medical device(s) indicated for dispensation by the code, with a request for the user to confirm that the displayed medical device(s) corresponds to the user's expectations.

The command may be made by the controller 150 for a particular locker 110, or may be for a particular device being held in one or more lockers 110. For example, the information derived from the received code may specifically identify a particular locker 110 as containing the contents of the user's order. This specific identification may be included during the initial generation of the code. In this example, the controller 150 retrieves this specific identification and commands the identified locker to open. In another example, the controller 150 may compare the particular device (or devices) identified by the received code to an inventory list of lockers 110 with their contents, and may determine a locker (or lockers) that has contents matching those of the information associated with the received code. The controller 150 may then issue a command to open the locker (or one of the lockers) with matching contents.

In some embodiments, the locker system 100 may be associated with a backend computing system that may track the inventory in the locker system 100, generate, store, and erase unique retrieval codes associated with specific locker/medical device combinations. The backend computing system may be in electronic communication with the locker system 100. In some embodiments, the backend computing system may similarly operate a plurality of locker systems 100 disposed at different medical providers.

Referring to FIG. 2, which shows a system 200 that includes the locker system 100, the locker system 100 may be in communication with a provider computing system 210, a user computing system 220, and a third party computing system 230. Additionally, each of the systems 210, 220, and 230 may be in communication with each other, such that each of systems 100, 210, 220, and 230 may be in communication with each of the other systems 100, 210, 220, and 230. The communication channels between each of the systems 100, 210, 220, and 230 may be encrypted and suitably-protected to comply with any applicable regulations.

The provider computing system 210 may be associated with a provider (e.g., doctor, prescriber, nurse practitioner, etc.), and may be in communication with the locker system 100 to place an order and to receive information regarding the order. For example, the provider computing system 210 may initiate an order with the locker system 100 by sending information regarding a patient, a prescription, a medical device, and/or payment data, and may receive order status data from the locker system 110 (e.g., whether the order is ready for pickup, whether the order has been picked up, whether additional data is required for the order, etc.).

The user computing system 220 may be associated with a user of the locker system 100. For example, the user computing system 220 may be a mobile device of a patient with an order being serviced by the locker system 100. Accordingly, the user computing system 220 may be configured to receive a code generated by the locker system 100, and may provide that same code to the locker system 100 as part of picking up an order. The user computing system 220 may also respond to prompts from the locker system 100, and may communicate with one or both of the provider computing system 210 and the third party computing system 230 to provide information as necessary.

The third party computing system 230 may be associated with a third party who may be involved in the order but is neither the patient nor the prescriber. For example, the third party may be an insurance company who may provide funding for the order, or the third party may be a guardian, parent, or otherwise legal representative for the patient. Accordingly, the third party computing system 230 may provide authorization and/or approval for the order. In some embodiments, an order may proceed, and a locker may open, only after (e.g., prior to and/or in response to) approval being received from the third party computing system 230.

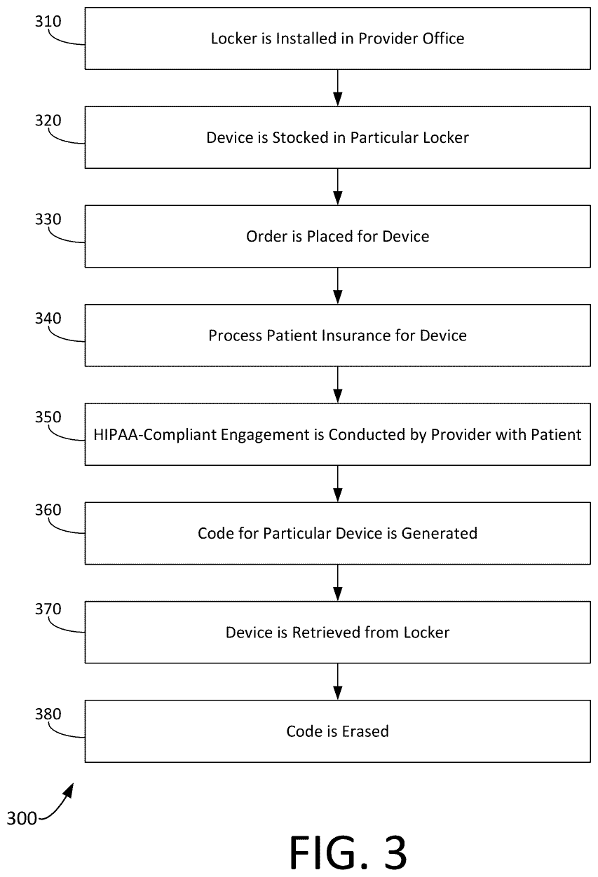

FIG. 3 is a flowchart of an example method 300 for distribution of a medical device with a secure locker system. The method 300 may include, at block 310, installing a locker system (e.g., secure locker system 100) in an office of a medical provider. Installation of the locker may be performed as part of (or at the culmination of) a registration process that begins with the medical provider joining a third party network. This third party network may be a supplier of medical devices, and may be responsible for stocking the secure lockers. This third party may be, for example, a locker system operator that is distinct and separate from the medical care provider.

The method 300 may also include, at block 320, stocking the secure lockers with medical devices. As discussed, these medical devices may be high-cost medical devices (e.g., LARC). In some embodiments, each medical device is specifically associated with the locker in which the device is stored, such that particular devices can be tracked and logged. For example, when a request is made for a device, a particular device is selected and dispensed, and because the system associates a device with a locker, the system can determine which device (e.g., by serial number) is dispensed. The locker system may be stocked by the locker system operator.

The method 300 may further include, at block 330, receiving an order for a device. This order may be received from a provider on behalf of a patient, or may be made by the patient directly. The order may be received over a network in communication with the secure locker system, or may be directly input into the system (e.g., via the I/O device 130). The order may be received by the locker system operator. In some embodiments, the order may be received with consents from both the patient and the medical care provider for the locker system operator to process the order.

The method 300 may include, at block 340, receiving approval for the order. In some embodiments, receiving approval for the order may include processing insurance for the order. In these embodiments, the insurance processed for the order may be the patient's insurance. If insurance is verified (e.g., the patient's insurance will cover payment for the device), the patient's information may be entered into a secure portal for tracking orders. An internal prescription may be generated to serve as a record for this verification. In some embodiments, particularly those in which the patient is uninsured or insufficiently-insured, the received approval may be from the patient themselves, and may be an approval of a cost associated with the order. Block 340 may be performed by the locker system operator.

The method 300 may include, at block 350, facilitating a tele-health engagement between the patient and the medical care provider. The engagement may be a text conversation between the provider and the patient, and may be facilitated by a HIPAA-compliant software. As part of the engagement, the provider may confirm the patient's choice of medical device, and may provide additional details re: the medical device (e.g., insertion of the LARC). The locker system operator may facilitate the engagement by providing a software platform through which the patient and the provider may communicate (e.g., via text message). The locker system operator may additionally or alternatively facilitate the engagement by indicating to the provider and/or patient that the patient's claim has been processed and prompting the patient and/or provider to initiate the engagement. This indication may include sending a message to the provider indicating a status of the claim, or may include a message to the provider or patient that includes a code to be input to the locker system to retrieve the associated order. The type of indication may be based on a status of the order. For example, if the order is complete and a locker is stocked with items from the order, the indication may be to the patient and include a code for pickup. In another example, if the order is complete but the locker is not yet stocked, the indication may be to the provider and may reflect the order status.

The method 300 may also include, at block 360, generating a unique retrieval code for the order, and, at block 370, distributing the unique retrieval code to one or more parties. The parties may be, for example, a patient, a provider, and/or a locker system. Accordingly, as a result of block 370, one or more of a patient computing system, a provider computing system, and a locker system may receive the unique retrieval code. This code may be a QR code, a bar code, a numerical code, or any other identifier that can be associated with the order. The code may be indicative of a particular medical device of the order and of approval by the provider, the patient, and/or the insurance. In those embodiments in which a particular device is associated with the particular locker in which the device is stored, the code may also include an indication of the particular locker. In some embodiments, the unique code for a given medical device/locker combination may be generated at the time the medical device is stocked in the locker. In other embodiments, the unique code may be generated in response to the order, and may be communicated to the provider via a secure portal (e.g., the portal used to initially place the order). In this embodiment, the code may be passed onto the patient by the provider (e.g., via a secure interaction between the provider and patient). In another example, the code may be provided to the patient directly via an email address on file and associated with the patient. In those instances in which the code is sent directly to the patient (e.g., not via a secure portal), the locker system may indicate such in the internal system and may require additional identifying information at pickup to maintain safety and to ensure that the user is the patient.

The method 300 may further include, at block 370, receiving the unique retrieval code at or by the locker system. The code may be received using a scanner (e.g., optical element 120) user input interface (e.g., I/O device 130) of the locker system. Once received, the code may be transmitted to a backend system (e.g., controller 150). The backend system may then compare the unique retrieval code to the retrieval codes associated with the various lockers of the locker system and, in response to the received code matching a code for one of the lockers, send a command to open the matched locker for the provider to retrieve the medical device. For example, the code may include an indication of a particular locker (e.g., encoded within the code), or the code may include an indication of a content of the order, and the locker system may determine which locker to open based on a comparison of the order contents with an inventory list.

The method 300 may include, at block 380, erasing the code from the locker system. This erasure may be performed in response to the locker opened in block 370 being closed. Erasing the code may prevent re-use, which provides an additional layer of security. In conjunction with erasing the code, the order may be marked as fulfilled.

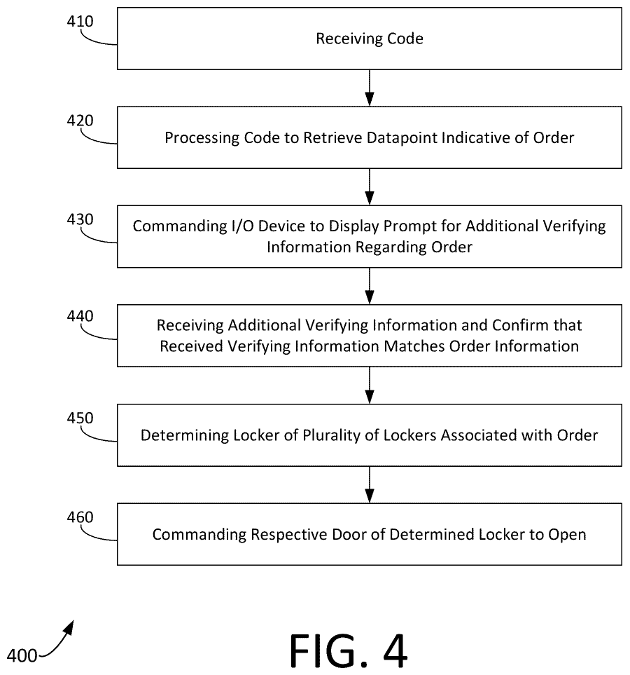

FIG. 4 is a flowchart of an example method 400 for distribution of a medical device with a secure locker system. One or more portions of the method 400 may be performed by the locker system 100 and, in particular, the controller 150.

The method 400 may include, at block 410, receiving a code from a user. The code may be a bar code (e.g., QR code) or numerical code, and may be received via a user mobile device (e.g., user computing system 220) and/or via an input device included with the locker system 100 (e.g., I/O device 130).

The method 400 may also include, at block 420, processing the received code to retrieve a datapoint indicative of an order associated with the code.

The method 400 may also include, at block 430, commanding an I/O device (e.g., I/O device 130) included with the locker system to display a prompt for additional verifying information regarding the order in response to determining the order at block 420. This additional verification information may include one or more datapoints that would satisfy a “Something-You-Know” factor test, such as a birthday of the patient, an address of the patient, etc. The information requested at block 430 may be selected from information categories stored in association with the order that is associated with the received code.

The method 400 may also include, at block 440, receiving additional verification information from the user in response to the prompt at block 430 and confirming that the received verification information matches the order information. If the information does not match, the user's scan of the unique code may be rejected and the code may be disabled. Alternatively, if the information does not match block 430 may be repeated to prompt the user for different verification information.

The method 400 may also include, at block 450, determining a locker of the plurality of lockers that is associated with the order, in response to the additional verification information matching order information. In some embodiments, the code data may indicate a specific locker associated with the order. In some embodiments, the code data may indicate a medical device associated with the order, and the controller 150 may determine an appropriate locker based on a comparison of the indicated medical device with an inventory list for the plurality of lockers.

The method 400 may further include, at block 460, commanding a respective door of the determined locker to open. Block 460 may be performed in response to confirming that the received verification information matches the order information at block 440, in some embodiments.

While this disclosure has described certain embodiments, it will be understood that the claims are not intended to be limited to these embodiments except as explicitly recited in the claims. On the contrary, the instant disclosure is intended to cover alternatives, modifications and equivalents, which may be included within the spirit and scope of the disclosure. Furthermore, in the detailed description of the present disclosure, numerous specific details are set forth in order to provide a thorough understanding of the disclosed embodiments. However, it will be obvious to one of ordinary skill in the art that systems and methods consistent with this disclosure may be practiced without these specific details. In other instances, well known methods, procedures, components, and circuits have not been described in detail as not to unnecessarily obscure various aspects of the present disclosure.

What is claimed is:

1. A system for secure lockers for dispensing medical devices, the system comprising:
a plurality of lockers, each of the plurality of lockers having a respective door;
an I/O device; and
a controller in communication with each of the plurality of lockers and to the I/O device, the controller configured to:
receive a code;
process the code to retrieve a datapoint indicative of an order;
command the I/O device to display a prompt for additional verifying information regarding the order;
in response to receiving additional verifying information, determine a locker of the plurality of lockers associated with the order; and
command the respective door of the determined locker to open.

2. The system of claim 1, further comprising an optical element in communication with the controller, wherein the controller receives the code via the optical element.

3. The system of claim 2, wherein the optical element is configured to, prior to the controller receiving the code from the optical element:
partially process the code to derive a label of the datapoint;
determine, based on the label, that the code is relevant; and
in response to the determine of relevancy, transmit the code to the controller.

4. The system of claim 1, wherein the controller receives the code via the I/O device.

5. The system of claim 1, wherein the code comprises a bar code or a numerical code.

6. The system of claim 1, wherein the additional verifying information comprises personal information respective of a patient associated with the order, the personal information satisfying a Something-You-Know factor test.

7. The system of claim 1, wherein the controller is further configured to:
prior to commanding the respective door, send a prompt for approval to a third party; and
in response to receiving approval from the third party, command the respective door to open.

8. A system comprising:
a processor; and
a computer-readable medium storing instructions that, when executed by the processor, cause the system to:
receive a code;
process the code to retrieve a datapoint indicative of an order;
command an I/O device to display a prompt for additional verifying information regarding the order;
in response to receiving additional verifying information, determine a locker of a plurality of lockers associated with the order; and
command the determined locker to open.

9. The system of claim 8, wherein the system receives the code via an optical element.

10. The system of claim 9, wherein the optical element is configured to, prior to the system receiving the code from the optical element:
partially process the code to derive a label of the datapoint;
determine, based on the label, that the code is relevant; and
in response to the determine of relevancy, transmit the code to the system.

11. The system of claim 8, wherein the system receives the code via the I/O device.

12. The system of claim 8, wherein the code comprises a bar code or a numerical code.

13. The system of claim 8, wherein the additional verifying information comprises personal information respective of a patient associated with the order, the personal information satisfying a Something-You-Know factor test.

14. The system of claim 8, wherein the instructions further cause the system to:
prior to commanding the determined locker, send a prompt for approval to a third party; and
in response to receiving approval from the third party, command the determined locker to open.

15. A method for dispensing medical devices, the method comprising:
receiving, by a controller, a code;
processing, by the controller, the code to retrieve a datapoint indicative of an order;
commanding, by the controller, an I/O device to display a prompt for additional verifying information regarding the order;
in response to receiving additional verifying information, determining, by the controller, a locker of a plurality of lockers associated with the order; and
commanding, by the controller, a respective door of the determined locker to open.

16. The method of claim 15, wherein the code is received via an optical element in communication with the controller.

17. The method of claim 16, wherein the optical element is configured to, prior to the controller receiving the code from the optical element:
partially processing the code to derive a label of the datapoint;
determining, based on the label, that the code is relevant; and
in response to the determine of relevancy, transmitting the code to the controller.

18. The method of claim 15, wherein the code comprises a bar code or a numerical code.

19. The method of claim 15, wherein the additional verifying information comprises personal information respective of a patient associated with the order, the personal information satisfying a Something-You-Know factor test.

20. The method of claim 15, further comprising:
prior to commanding the respective door, sending a prompt for approval to a third party; and
in response to receiving approval from the third party, commanding the respective door to open.
